(19) United States
(12) Patent Application Publication
(10) Pub. No.: US 2020/0020423 A1
(43) Pub. Date: Jan. 16, 2020

(54) A METHOD AND SYSTEM FOR MATCHING SUBJECTS TO CLINICAL TRIALS

(71) Applicant: KONINKLIJKE PHILIPS N.V., EINDHOVEN (NL)

(72) Inventors: Qingxin Wu, Lexington, MA (US); Alexander Ryan Mankovich, Boston, MA (US); Abhishek Talluli, Eindhoven (NL); Kostyantyn Volyanskyy, Larchmont, NY (US); Nevenka Dimitrova, Pelham Manor, NY (US); Yong Mao, Hawthorne, NY (US); Charles Yee, Boston, MA (US)

(21) Appl. No.: 16/334,823

(22) PCT Filed: Sep. 26, 2017
(86) PCT No.: PCT/IB2017/055823

§ 371 Date: Mar. 20, 2019

Related U.S. Application Data
(60) Provisional application No. 62/401,313, filed on Sep. 29, 2016.

(51) Int. Cl. G16H 10/20; G16H 10/60; G06F 16/22

(52) U.S. Cl. CPC G16H 10/20; G06F 16/2272; G16H 10/60

(57) ABSTRACT

A system and method for providing a prioritized list of clinical trials that are relevant to a patient suffering from an illness or disease, such as cancer, are disclosed. Specifically, a method for conducting an automated, real time clinical trial search and a prioritization analysis is described. The method comprises the steps of conducting an automated full-text clinical trial search based on structuralization of clinical trial eligibility data and knowledge-based inference, initiating a query from the patient's side, and providing a prioritized list of all accessible clinical trials fulfilling a particular query. The system provides better sensitivity, precision and negative predictive value than the current most known clinical trial matching tool: clinicaltrials.gov.

### FIELD OF THE INVENTION

The present invention relates to a data-driven integrative system and method for providing a prioritized list of clinical trials that are relevant to a patient suffering from an illness or disease, such as cancer, are disclosed. Specifically, a method for conducting an automated, real time clinical trial search and a prioritization analysis is described. The method comprises the steps of conducting an automated full-text clinical trial search based on structuralization of clinical trial eligibility data and knowledge-based inference, initiating a query from the patient's side, and providing a prioritized list of all accessible clinical trials fulfilling a particular query. The system provides better sensitivity, precision and negative predictive value than the current clinical trial matching tool: clinicaltrials.gov.

### BACKGROUND OF THE INVENTION

Clinical trials are of vital importance in the treatment of many diseases, especially cancer, and are conducted under specific healthcare protocols. Many clinical trials fail because of the difficulty in enrolling a sufficient number of eligible patients in a short period of time. Clinical trial failures delay new treatments and therapies from reaching health care providers and also increase the health and economic burden for both patients and trial sponsors. Several computer-implemented clinical trial matching methods and systems have been described since 2010, but the implementation of these methods relied on specific trials and were not patient-centric or for personalized purpose. These methods, based on keyword matching systems, basically match query strings with pre-selected key words from trial documents, such as those found on Clinicaltrials.gov. Such methods have several important disadvantages. First, keyword searching cannot fully cover what's in the clinical trial document. Second, the identification of keywords itself is difficult and not necessarily accurate. Further, there are no semantic considerations in keyword-based methods.

Current solutions lack the capability of using patient-specific data to automatically compare with clinical trial criteria and identify relevant patient-specific trials to physicians, provided as a prioritized list of most relevant patient-specific clinical trials. Most current clinical trial matching systems do not have adequate statistical robustness (reflected by metrics such as sensitivity, specificity, precision, etc.), for searching clinical trials, since they are key-word based. A full-text index-based search engine will be much more prone to generate better results.

FIG. 1 shows the high-level infrastructure for a next-generation matching system that can use both clinical trial (“CT”) criteria and patient-specific data to suggest clinical trials that would be most suitable to a particular patient. This infrastructure is composed of two main parts: the first part is a CT matching system which translates structured patient-specific data into structured factors, such as disease, gene, and age, as input, and searches within clinical trial information databases to generate a list of matched trials. The second module performs the task of prioritizing the list based on pre-determined criteria, since eventually each patient will be mostly enrolled into one trial. Besides using natural language processing (“NLP”), and knowledge formalization to structure the patient information, how to search and prioritize clinical trials are very valuable aspects in the real practice along the workflow, assuming we have patient information structuralized already.

This invention addresses the clinical trial search and prioritization issue as a whole. Compared with the current publically available engines, e.g., clinicaltrials.gov, the system of this invention provides a better solution for matching patients with clinical trials that would be most beneficial to the patient. In this invention, we developed such a full-text engine and compared it with clinicaltrials.gov. After testing a set of cases covering multiple disease terms and other criteria, we find our engine improves upon the poor sensitivity, precision and negative predictive value exhibited by clinicaltrials.gov.

In further contrast, most of the systems currently available do not have comprehensive prioritization modules. In this invention, we use an index comprehensively considering term frequency, inverse document frequency and field-length normalization to provide prioritization of the relevant clinical trials.

### SUMMARY OF THE INVENTION

In particular, an object of the present invention is to provide a system and method that solves the above-mentioned problems of the prior art by determining and providing a prioritized list of relevant patient specific clinical trials. It is also an object of the present invention to provide a system and method for providing a list of relevant patient-specific clinical trials based on selected searching, structuralizing and matching criteria selected by a user of the system and method. It is a further object of the present invention to identify relevant patient-specific clinical trials based on patient medical status and logistical criteria. It is also an object of the present invention to provide an alternative to the prior art.

Thus, the above-described object and several other objects are intended to be obtained in a first aspect of the invention by providing a system and method for providing a prioritized list of relevant patient-specific clinical trials, such method comprising the steps of:

with a computing device with a graphical user interface, determining a dataset of full-text clinical trial documents by obtaining network documents for all active clinical trials, and storing said clinical trial documents as an XML, file on a server configured to store said dataset;

structuralizing said stored clinical trial documents by an XML, document parser unit,

indexing, by a mapping unit, said stored clinical trial documents,

determining a sub-network of said clinical trial documents from said dataset through searching said structured and indexed clinical trial documents based on structured clinical trial eligibility criteria, by performing at least one query search of said structured and indexed clinical trial documents;

inputting patient-specific data, by a user interface, onto a processor configured to receive said patient-specific data,

matching said patient-specific data with said structured and indexed clinical trial documents, according to selected matching criteria,

ranking the clinical trial documents identified by said matching step according to selected ranking criteria,

generating a list of said ranked clinical trial documents;

displaying said list of ranked clinical trial documents on a graphical user interface.

In addition, a second aspect of the present invention is directed to a non-transitory computer readable storage medium storing one or more programs for providing a prioritized list of relevant patient-specific clinical trials, the one or more programs comprising instructions, which when executed by a computing device with a graphical user interface, cause the device to carry out the steps of the method as described above.

### BRIEF DESCRIPTION OF THE DRAWINGS

The methods according to the invention will now be described in more detail with regard to the accompanying figures. The figures showing ways of implementing the present invention and are not to be construed as being limiting to other possible embodiments falling within the scope of the attached claims.

FIG. 1 is an overview of the within method and system for matching patients with ranked, eligible clinical trials;

FIG. 2 is a flowchart of the within keyword-based clinical trial matching method of the within invention illustrating a pathway of steps that of the method;

FIG. 3 illustrates an analyzer pipeline;

FIG. 4 is a block diagram of the ranking step of the within invention; and

FIG. 5 is a GUI web application example front end screenshot.

### DETAILED DESCRIPTION OF THE INVENTION

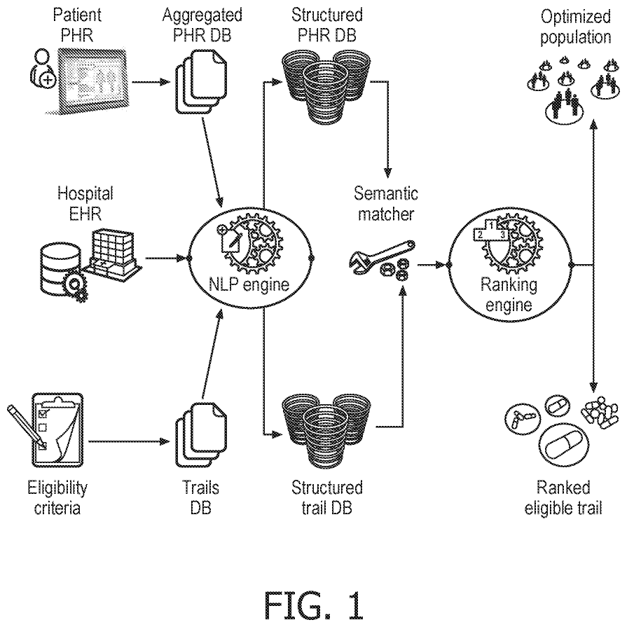

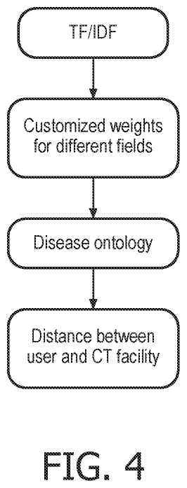

The present invention provides a system and method for providing a prioritized list of relevant clinical trials specific to a particular patient, by integrating automated, real-time clinical trial data obtained from full-text searching using structuralized clinical trial eligibility criteria and knowledge-based inference, and patient side query based on patient-specific data. The present invention is described in further detail below with reference made to FIGS. 1-5.

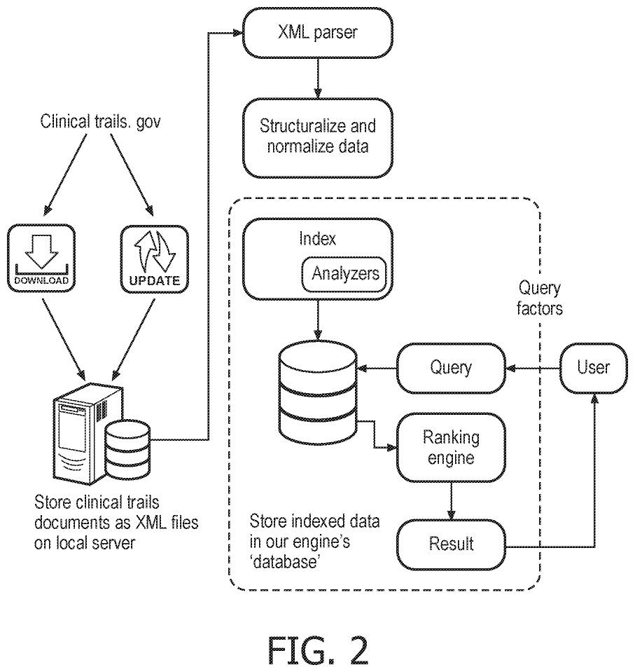

According to an embodiment of the present invention, FIG. 1 is an overview of the within method and system for matching patients with ranked, eligible clinical trials. A flowchart presenting the overall block-diagram of the method for providing patient-specific prioritized relevant clinical trial matches, is set forth by the steps, or modules, outlined in FIG. 2. The first step, or module, includes downloading and maintaining the most update-to-date clinical trials database(s). Downloading the clinical trials database is necessary before conducting any searches. Each clinical trial is stored as an xml file on a local server. A continuous stream of incoming clinical trials from, e.g., clinicaltrials.gov, is regularly maintained and the database is constantly updated with new trials.

Structured patient data for the method of the invention including; age, gender, gene, amino acid substitution (genomic data), cancer stage, tumor grade and disease diagnosis, is inputted and stored on a database. More broadly genomic information can include any gene expression, gene fusions, DNA methylation, histone modifications, protein expression metabolomic data. Further patient information includes; patient medical conditions, manifestations, medications, therapy/surgery, and other relevant medical, quantitative self information. This data is structured and normalized. This structaralization could be enabled by user entry or fully automated parsing of clinical IT data, by an HL7 broker engine.

Next, the CT data is structuralized and normalized. For this purpose, an XML document parser is used to parse the stored clinical trial documents and extract useful information, such as the CT design, eligibility criteria and geographical/location details. For comparing patient information with criteria from CT, further CT information needs to be normalized, such as age, cancer stage and tumor grade, since they may occur in many different formats. For example, the cancer stage can be ‘stage I,II,III,’ ‘stage I-III,’ ‘stage I or III,’ ‘stage I and III,’ etc. This information would be normalized into a canonical format such as ‘stage I, stage II, stage III’ before being stored in the database. Minimum age and maximum age are normalized to minutes. If not found, the minimum age is set to 0 and maximum age is set to 200*365*24*60.

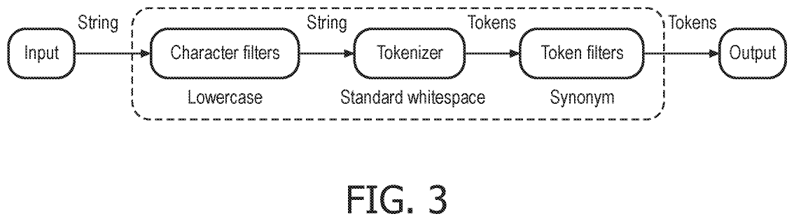

Once the CT information is stored and structured, it is then indexed. Indexing (or mapping) is the process of defining how a document, and the fields it contains, is stored and indexed. This is done by the “analyzer,” an important component of index definition. See FIG. 3, showing the pipeline for the analyzer. For each section of a CT document, the strings are first lowercased and tokenized using a built-in standard tokenizer and whitespace tokenizer. Then, gene terms, which are often found in the CT document's eligibility criteria section, are passed through a synonym filter where the canonical expression is returned. Because synonyms are very common for genes (for example, ‘tp53’ is one synonym of ‘p53’), incorporating synonyms into the analyzer tool significantly increases the number of potential matches of clinical trials. Gene synonyms (including family names, aliases, previous names, and previous symbols) are obtained from public databases. Similarly, a synonym filter for the disease name is also incorporated to further improve the performance of the trial matching engine when queries also involve disease diagnosis.

The present invention uses an inverted index, a structure which is designed to allow fast full-text index and query, a very important concept in full-text searching. An inverted index consists of a list of all the unique words that appear in any document, and is an index data structure storing a mapping from content, such as words or numbers, to their locations in a document or a set of documents. It is named in contrast to Forward Index, which maps from document to content. For example,

‘hello’: doc1:1, doc3:10 (docid: position)

‘world’: doc1, doc2, doc3 (docid)

For each word, via the hash table or the index we find a list of the documents in which the word appears. This mechanism allows much faster searching than matching each term in each document.

Real world query usually involves multiple factors, including, e.g., disease name, gene, mutation type, gender, age, cancer stage, and tumor grade. A query module builds the query to interact with the clinical trial data base on query factors provided by the user through a user interface. The within invention uses a Boolean model to find matching documents, and a formula called the practical scoring function to calculate relevance. Bool query matches documents by matching Boolean combinations of other queries. The Boolean model simply applies the AND, OR, and NOT conditions expressed in the query to find all the documents that match. For example:

```
{
  “query”: {
    “bool”: {
```

| “must”: | [ | |
|---|---|---|
| | | { “match”: { “purpose”: “lung cancer”, “operator”:“and”}}, |
| | | { “match”: { “inclusion criteria”: “egfr”} } |

]

| | |
|---|---|
| “must_not”: | { “match”: { “exclusion criteria”: “pregnant” }}, |

```
      “should”: [
        { “match”: { “title”: “tumor” }} ]
    }
  }
}
```

This is a bool query that has must query, must query and should query combined together. It defines that:
‘lung’ and ‘cancer’ must appear in field ‘purpose’
AND
‘egfr’ must not appear in field ‘inclusion criteria’
AND
‘pregnant’ must not appear in field ‘exclusion criteria’
Any documents that meet the logical statements above will be a match. ‘Should’ match will not affect the bool query result, but if a document meets this criteria, it will have higher score. This process is simple and fast, excludes any documents that cannot possibly match the query.

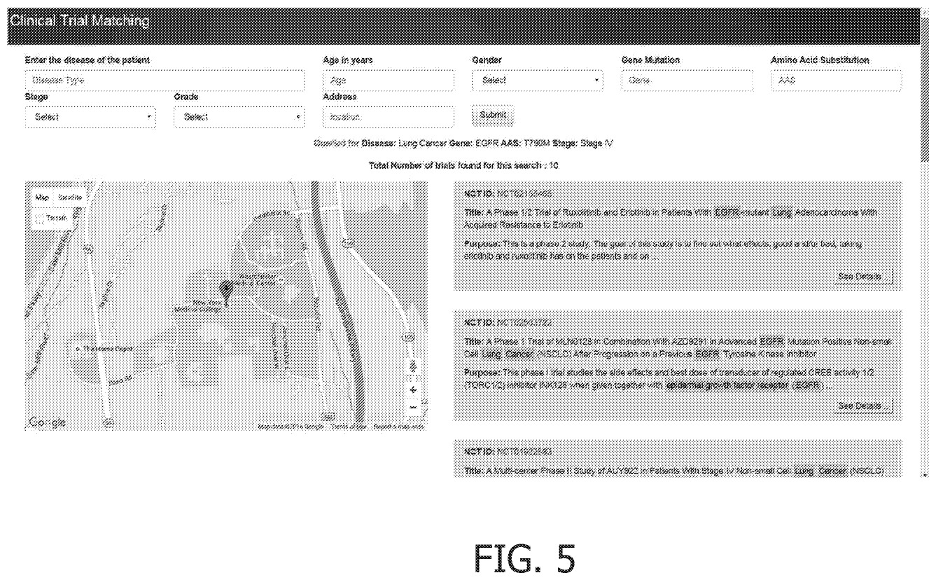

Referring to FIG. 5, once a list of matching documents is identified that meets the evaluation of our Boolean model, i.e., the documents meet the search query criteria, the documents are ranked by relevance. First, our invention uses Lucene's practical scoring function to calculate the score of each matched document, which is:

$$score ( q , d ) = {\sum }_{t ∈ q} ( tf ( t , d ) \cdot {idf ( t )}^{2} \cdot t . getBoost ( ) \cdot norm ( t , d ) )$$

where, score(q,d) is the relevance score of document d for query q;
the summation part calculates the sum of the weights for each term t in the query q for document d.
tf(t,d) is the term frequency for term tin document d.
idf(t) is the inverse document frequency for term t.
t.getBoost( )is the boost that has been applied to the query.
Norm(t,d) is the field-length norm, combined with the index-time field-level boost.

The concept is that the relevance score of the entire CT document depends (in part) on the weight of each query term that appears in that document. Term frequency, inverse document frequency, and field-length norm are used together to calculate the weight of a single term in a particular document. These are calculated and stored at the time of indexing. Queries usually consist of more than one term. This invention uses a vector space model to combine the weights of multiple terms.

In query's definition, extra weight can be given to each field. Because, within a CT document, not all sections have equal importance, such as brief title should be more important than detailed description, section/field's weight is tuned for relevance at the time of query. Weights are assigned for each field thus, when calculating score, a term that occurs in a field with weight 2 will get twice the score than the same term that occurs in a field with weight 1, i.e., a field with weight two is twice as important as the field with weight one.

Besides the scoring methods, many other factors are considered in the clinical trial matching prioritization process, e.g., trial costs, trial location's distance to a patient's home address, as well as extensive overview pertaining to the patient's vital signs and profile. As an open ranking system, the within system provides user interfaces to further expand prioritization functions based on these and other factors. In other words, after getting the list of documents that are scored, they are input into a rank engine to do post-ranking. For example, the invention utilizes two kinds of post-ranking. The first post-ranking criteria relates to disease ontology. The second post-ranking criteria relates to between the patient and the facility that provides the specific clinical trial.

The system of this invention further provides a graphic user interface (“GUI”) to enhance the user's experience and also to allow the user to input criteria, select further information and view the prioritized list of relevant clinical trials. Web applications, like the one developed using Django for the within clinical trial matching engine method, significantly improve the end user's experience. Users may provide search queries to the web application and quickly visualize the matching trials, where matched terms are highlighted. Referring to FIG. 5, A Google map adjacent to the trial matches enables the patient to choose trials based on geographical proximity to a home or treatment center. It also facilitates integration into other projects.

The performance of described system was evaluated by using clinicaltrials.gov as a comparison. Several metrics were employed for evaluating the performance: sensitivity, specificity, positive predictive value and negative predictive value. A true positive was defined as a trial that rendered a match to terms contained in the query. A false positive was defined as a trial which did not render a complete match. As true negatives are difficult to ascertain without significant resources (there are about 46,000 open clinical trials), these trials were those which one solution (e.g., clinicaltrials.gov) incorrectly returned (i.e., false positives), but which the method of the invention the invention, correctly, did not return. False negatives, conversely, were trials that one solution (e.g., clinicaltrials.gov) correctly returned (i.e., true positives) but which the method of the invention, incorrectly, did not return. Overall, the method of this invention increased performance for every statistical metric apart from specificity: increased sensitivity by 40%; decreased specificity by 7%; increased the positive predictive value (i.e., precision) by 4%; and increased the negative predictive value by 21%.

### EXAMPLE 1

### Clinical Expert Embodiment

In one embodiment of the invention, as an example, a medical oncologist, Dr. A, wants to find a suitable clinical trial for treating his patient, B, with late-stage cancer. Dr. A would use this method to directly access all available clinical trials. Because our system is synchronous with clinical trial information sources, all related CT information is up-to-date. An inner index instance is maintained to store the inverted indexed clinical trial information based on the sections extracted by xml parser. With the GUI developed, Dr. A is able to search matching clinical trials by typing in patient information, including, e.g., disease diagnosed, age, gender, disease stage and grade. The semantic capability of the method will enhance its matching capability. Thus, if the patient, B, is diagnosed with a brain tumor), clinical trials related to glioblastoma multiform (GBM, one type of brain tumor) will also be identified as potential matches. With a list of matched clinical trials reported from the matcher, the ranking engine further prioritizes these trials according to the default conditions or those set by the user using the aforementioned method.

### EXAMPLE 2

### Tumor-Specific Wide-Breadth Mutational Queries Embodiment

As an example of a further another embodiment, Dr. A begins with patient B's genomic aberrations. If whole exome (or targeted exome, with a subset of gene regions relevant to cancer) sequence data is available, Dr. A first matches the somatic mutations from B's tumor to the mutations saved in the index made with all clinical trials' documents via the within method. With a list of matched clinical trials reported, the ranking engine could further prioritize these trials according to distance between the patient's home address and clinical trial sites.

1. A computer-implemented method for providing a prioritized list of relevant patient-specific clinical trials, the method comprising:
a computing device with a graphical user interface,
determining a dataset of full-text clinical trial documents by obtaining network documents for all active clinical trials, and storing said clinical trial documents as an XML file on a server configure to store said dataset;
structuralizing said stored clinical trial documents by an XML document parser unit,
indexing, by a mapping unit, said stored clinical trial documents,
determining a sub-network of said clinical trial documents from said dataset through searching said structured and indexed clinical trial documents based on structured clinical trial eligibility criteria, by performing at least one query search of said structured and indexed clinical trial documents;
inputting patient-specific data, by a user interface, onto a processor configured to receive said patient-specific data,
matching said patient-specific data with said structured and indexed clinical trial documents, according to selected matching criteria,
ranking the clinical trial documents identified by said matching step according to selected ranking criteria,
generating a list of said ranked clinical trial documents;
displaying said list of ranked clinical trial documents on a graphical user interface.

2. A non-transitory computer readable storage medium storing one or more programs for providing a prioritized list of relevant patient-specific clinical trials, the one or more programs comprising instructions, which when executed by a computing device with a graphical user interface, cause the device to carry out the steps of the method as defined in claim 1.

3. A method for providing a prioritized list of relevant patient-specific clinical trials, said method comprising the steps of:
determining a dataset of full-text clinical trial documents by obtaining network documents for all active clinical trials;
structuralizing said clinical trial documents,
indexing said clinical trial documents,
determining a sub-network of said clinical trial documents from said dataset through searching said structured and indexed clinical trial documents based on structured clinical trial eligibility criteria, by performing at least one query search of said structured and indexed clinical trial documents;
inputting patient-specific data onto a processor configured to receive said patient-specific data,
matching said patient-specific data with said structured and indexed clinical trial documents, according to selected matching criteria,
ranking the clinical trial documents identified by said matching step according to selected ranking criteria,
generating a list of said ranked clinical trial documents;
displaying said list of ranked clinical trial documents on a graphical user interface.
